(19) United States
(12) Patent Application Publication
(10) Pub. No.: US 2021/0275756 A1
(43) Pub. Date: Sep. 9, 2021

(54) VEIN STABILIZER DEVICE FOR THE VENIPUNCTURE PROCESS

(71) Applicant: Brent Joseph Halenkamp, Trinity, FL (US)

(72) Inventor: Brent Joseph Halenkamp, Trinity, FL (US)

(21) Appl. No.: 17/249,536

(22) Filed: Mar. 4, 2021

Related U.S. Application Data
(60) Provisional application No. 62/985,029, filed on Mar. 4, 2020.

(51) Int. Cl. A61M 5/42; A61B 5/15

(52) U.S. Cl. CPC A61M 5/425; A61B 5/150801

(57) ABSTRACT

A vein stabilizer device for venipuncture procedures is provided. The vein stabilizer device is embodied in an elongated body providing an external stabilizer tip protruding from a distal end of the body. The stabilizer tip may be defined by a supratip break, an infratip break, and two lateral breaks. Surface area of the breaks are dimensioned and adapted to engage the skin of a patient so as to stabilize a vein therein. The elongated body has a cavity for slidably receiving a finger of a user in such a way that the user can selectively apply pressure through a chosen break surface.

### CROSS-REFERENCE TO RELATED APPLICATION

This application claims the benefit of priority of U.S. provisional application No. 62/985,029, filed 4 Mar. 2020, the contents of which are herein incorporated by reference.

### BACKGROUND OF THE INVENTION

The present invention relates to medical devices for the administration of an intravascular needle and, more particularly, a vein stabilizer device for the venipuncture process.

In medicine, venipuncture is the process of obtaining intravenous access for the purpose of venous blood sampling or intravenous therapy. Finding a suitable vein can be a challenge. On top of that, successfully accessing an identified vein can be even more difficult. One of the biggest reasons practitioners can miss the vein is due to “rolling”—a rolling vein is a vein that moves during the process of obtaining intravenous access.

Vein stabilizers, however, are not used in the medical industry. Patients now are getting stuck multiple times due to nurses not being able to stabilize the vein. As the patient is usually already experiencing health problems, having to endure the practitioner struggling to successfully hit a vein, a seemingly rudimentary proposition, can exponentially add stress to the patient. Nurses are also at risk with being stuck by the needle due to the primitive venipuncture methods currently practiced.

Other devices in the field venipuncture methods are inefficient and ineffective, and do not stabilize the identified vein, nor apply pressure at the location of the vein. These are device are also uncomfortable and unsafe for the patient as well as the nurses. Moreover, these devices do not protect the nurses from accidental self-induced punctures. Finally, the current solutions are not only time consuming to use and take a lot of space, they are complex and expensive, which can intimidate a patient, yet another source of unwanted stress.

As can be seen, there is a need for a vein stabilizer device for the venipuncture process. The vein stabilizer device is dimensioned and adapted to slidably receive any finger of a user. The vein stabilizer device provides a multi-sided exterior having specific ridges and stabilizer protrusions adapted to engage, apply pressure to, and stabilize a vein. The vein stabilizer device is made of a material sufficient to protect the user's finger(s) from being stuck with a needle, ultimately protecting the user and comforting the patient. In short, the vein stabilizer device embodied by the present invention provides the following improvements: procedure, shape, safety, size, comfort, durability, effectiveness, efficiency, cost, and space.

### SUMMARY OF THE INVENTION

In one aspect of the present invention, a vein stabilizer device for venipuncture procedures, the device includes the following: a body; an external stabilizer tip protruding from a distal end of the body, wherein the stabilizer tip is defined by a supratip break and an opposing infratip break and at least one lateral tip break; and each tip break is configured to engage skin of a patient so as to stabilize a vein therein, wherein the stabilizer tip is further defined by at least one lateral break, wherein the body has an internal cavity communicating with an opening at a proximal end of the body, and wherein a taper along an upper surface of the cavity tapers upward as it extends toward the stabilizer tip, wherein a length of the cavity is dimensioned and adapted to extend a full length of a human finger, and wherein the body is made of puncture resistant material.

In another aspect of the present invention, method of improving a venipuncture procedure, the method includes the following: providing the above-mentioned vein stabilizer device; sliding a finger in the cavity; and selectively applying pressure to a skin surface through a surface area of one of the breaks in such a way to stabilize a vein under the skin surface.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a top perspective view of an exemplary embodiment of the present invention;

FIG. 3 is a bottom perspective view of an exemplary embodiment of the present invention;

FIG. 4 is a top plan view of an exemplary embodiment of the present invention;

FIG. 5 is a side elevation view of an exemplary embodiment of the present invention; and

FIG. 6 is a section view of an exemplary embodiment of the present invention, illustrating, taken along 6-6 in FIG. 2.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a vein stabilizer device for venipuncture procedures. The vein stabilizer device is embodied in an elongated body providing an external stabilizer tip protruding from a distal end of the body. The stabilizer tip may be defined by a supratip break, an infratip break, and two lateral breaks. Surface area of the breaks are dimensioned and adapted to engage the skin of a patient so as to stabilize a vein therein. The elongated body has a cavity for slidably receiving a finger of a user in such a way that the user can selectively apply pressure through a chosen break surface.

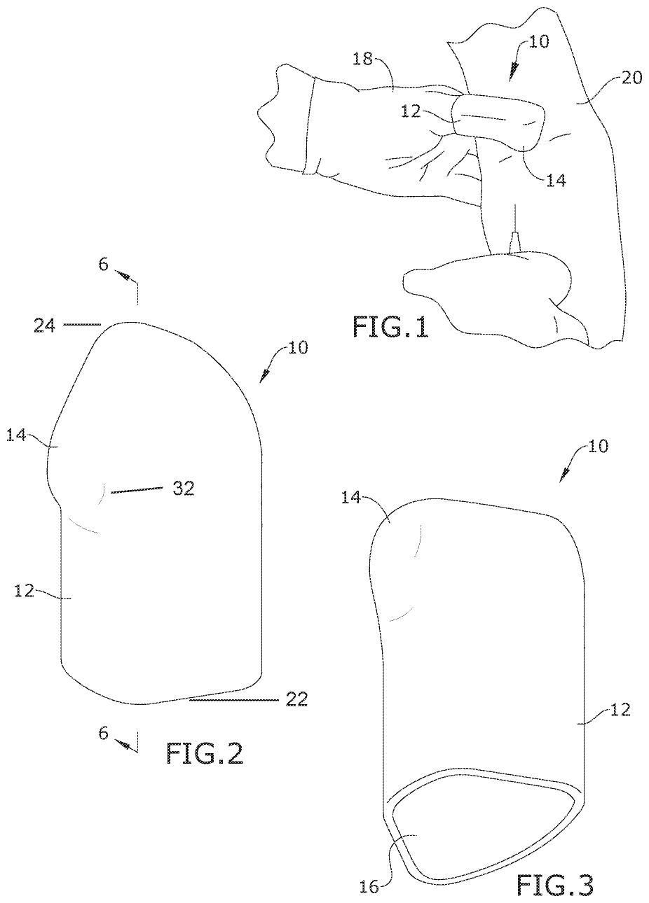

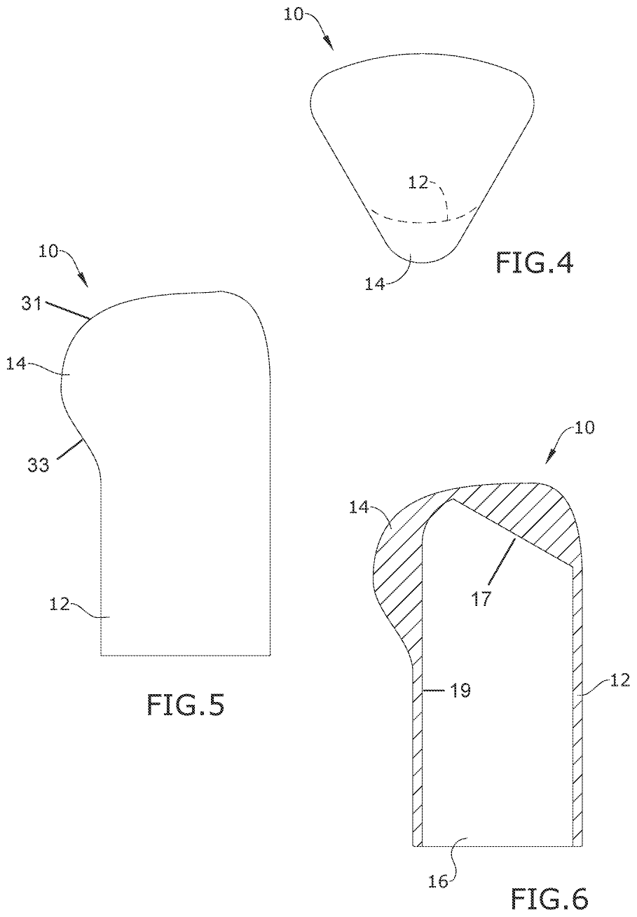

Referring now to FIGS. 1 through 6, the present invention may include a vein stabilizer device 10. The vein stabilizer device 10 may have a hollow elongated body 12. The body 12 may extend from a proximal opening 22 to a distal tip 24. The proximal opening 22 communicates to the cavity 16 dimensioned and adapted to slidably receive a finger of the user 18. The height and width of the body 12 and the cavity 16 are large enough to accommodate all finger sizes, wherein the length defined by a distance between the proximal opening 22 and the distal tip 24 are dimensioned to protect/shield the length of the finger. Furthermore, the material of the body 12 is puncture resistant so as to protect the user 18 from accidental needle sticks.

The cavity 16, as shown in FIG. 6, provides a taper 17 along an upper interior surface of the cavity 16 to accommodate all finger sizes. The taper 17 tapers upward as it extends toward the front stabilizer tip 14. The taper 17 in concert with the interior sidewall 19 of the cavity 16 at the fingertip enables a more secure engagement of the finger to enable more leverage.

It is understood that the directional terms, such as upper, upward, top, lower, bottom, downward. and the like are made in reference to FIG. 6, wherein the direction toward the top of FIG. 6 is deemed upward, while the direction toward the bottom of FIG. 6 is deemed downward.

The stabilizer tip 14 is deemed along the front of the body 12. The stabilizer tip 14 at the head of the vein stabilizer device 10 protruding outward relative to the remainder of the front of the body 12, thereby the stabilizer tip 14 is adapted to apply enough pressure to anchor a vein in order to prevent the vein from rolling.

The stabilizer tip 14 may have an apex that squarish, offering supratip break 31 and an infratip break 33, illustrated in FIG. 5. as well as lateral breaks 32, as illustrated in FIG. 2, each offering a unique surface to through which to apply pressure to the patient's vein.

The stabilizing tip 14 can extend between ⅛th inch to ⅝th inch in length from the exterior surface of the body 12 (past the infratip break 33) in order to apply sufficient pressure to anchor the vein in place. The stabilizing tip 14 itself can be squarish or rectangular in cross-sectional area at its apex or most prominent portion, where the rectangle can be defined by sides having lengths that range from 1/16 of an inch to three-quarters of an inch. The body 12 may extend from a proximal opening 22 to a distal tip 24 can be one-and-a-half inches to four-and-a-half inches in length to protect the full length of the finger. The height ⅞th of an inch to two inches and width one inch to on and ⅝ inches of the body 12 and the cavity 16 are large enough to accommodate all finger sizes.

In operation, the user 18 places their finger(s) into the cavity 16 and ties a tourniquet on the patient's arm. When the vein is showing through the pressure of the tourniquet, then the user 18 places the stabilizer tip 14 on top of the vein of the patient 20, and applies stabilizing pressure to the vein, preventing it from rolling. The user 18 can switch to one finger or both fingers for more grip, pressure and/or comfort. From there the user 18 then takes the needle and applies the venipuncture procedure. This will comfort and keep safe both the user 18 as well as the patient 20.

A method of making the present invention may include the following. A manufacturer could use additive manufacture, injection molding or other manufacturing processes as long as the resulting vein stabilizer device 10 functions as disclosed herein. One method of manufacture includes making the present invention by uploading the CAD design on a computer. From there, the CAD design may then cut out by the molding machine to make the injection mold for mass production. When the injection mold is made, then the mass production phase begins by injecting plastic to make the unique design of the present invention.

The injection molding is used in order to produce a specific design for the venipuncture device. This can be replicated is by multiple variations such as the following: clay, metal, rubber other plasticized materials, or other durable materials.

From the process of making the injection molding or cutting it out by hand, the design can be replicated by hand or by machine using either a CAD design, or any drawing. Also, another replication is the different shapes around the device. Instead of the ridges from the original concept, other variations and shapes can be added in order to differ the object from the original concept.

A method of using the present invention may include the following. The vein stabilizer device 10 disclosed above may be provided. A user 18 may place the vein stabilizer device 10 on one or their fingers. From there the user 18 can determine which side of the vein stabilizer device 10 and/or the stabilizer tip 14 they want to apply depending on the patient's vein and comfort. After the desired side has been chosen by the user in accordance with the patient's vein, then pressure is applied with the vein stabilizer device 10 through the applicable stabilizer tip 14 to stabilize the vein. At which time, the procedure of venipuncture is continued.

Additionally, the present invention can be used by a computer device instead of an actual human nurse. If the arm is placed within a machine that detects the vein and stabilizes the vein, then the computer can perform venipuncture.

It should be understood, of course, that the foregoing relates to exemplary embodiments of the invention and that modifications may be made without departing from the spirit and scope of the invention as set forth in the following claims.

What is claimed is:

1. A vein stabilizer device for venipuncture procedures, the device comprising:
a body;
an external stabilizer tip protruding from a distal end of the body, wherein the stabilizer tip is defined by a supratip break and an opposing infratip break; and
each tip break is configured to engage skin of a patient so as to stabilize a vein therein.

2. The device of claim 1, wherein the stabilizer tip is further defined by at least one lateral tip break.

3. The device of claim 2, wherein the body comprises an internal cavity communicating with an opening at a proximal end of the body.

4. The device of claim 3, further comprising a taper along an upper surface of the cavity, wherein the taper tapers upward as it extends toward the stabilizer tip.

5. The device of claim 4, wherein a length of the cavity is dimensioned and adapted to extend a full length of a human finger.

6. The device of claim 5, wherein the body is made of puncture resistant material.

7. A method of improving a venipuncture procedure, the method comprising:
providing a vein stabilizer device for claim 6;
sliding a finger in the cavity; and
selectively applying pressure to a skin surface through a surface area of one of the breaks in such a way to stabilize a vein under the skin surface.
